SEQUENCE LISTING (7 sequences)

SEQ ID NO: 1 - protein, 385 aa, Ipomoea batatas
MAAAAPSSSLLGVLMLLASIGYAAACYSAVFGFGDSLTDAGNLIRLEPDGTVPHMYFPPYGETYFDKPTGRCSDGRLIVDLIAQHYGLPLPPPSIPASLEGDKNRLRAGVNFAVVGSRALDAQFYEQRDIFDTVTNVSMTDQLNWFKELLPSLCSSPHDCKELLDGSLFVLGEFGGNDYTHSLLSGKGINVIRPFIPIVTRAIAQAVHELVELGARTVMIPSVLPLGCAASYLTYYPSPNPDDYDELGCLIWVNEMASYHNQLLQQQLAHVRELHPYANIVFADIYNAAMEVYQGPDTYGFSGGALRACCGGGGPYNFNTSAQCGNEGATACDNPSSYVNWDGYHLTEAAYQCITTGLLEGPYTYPRMKNLCRFDSRKTSRVAQI

SEQ ID NO: 2 - DNA, 1158 bp, Ipomoea batatas
atggccgccgcagctccttcttcctcgttgcttggtgtcctgatgctgttagcttccattggctacgccgccgcttgctattcggccgttttcggcttcggcgattccctcacggatgccggaaacctaatccgccttgagcctgacggcaccgtcccccacatgtacttcccgccctacggcgaaacctacttcgacaagcccaccggacgctgctccgacggccgcctcatcgtcgatttaatcgctcagcattacgggcttccgcttccgccgccttcgatcccggccagtttggaaggcgacaaaaaccgcctccgtgccggagtcaacttcgcggtggtcggatcacgtgcgctcgacgcgcaattctacgaacagagagacattttcgacactgtcaccaacgtttccatgacagatcagttgaattggttcaaggaattgctcccatctctgtgctcatcacctcacgattgcaaggagctattggatgggtcattgtttgtactaggagaatttggaggcaacgattacacgcactcgcttctatccggaaaaggtatcaatgtcatccgaccgttcattccaatcgtcacccgcgcaattgctcaggctgtccatgaattagttgagctaggtgcgaggacagtaatgatccccagcgtgttaccacttggatgtgcagcctcgtacttgacatactacccaagtcccaacccagatgactacgatgaactaggttgtctcatttgggttaacgaaatggcttcttaccacaaccaactcctccagcaacaactcgcccacgtaagagaacttcatccctatgccaatatcgtcttcgccgatatttacaatgccgccatggaggtttaccagggtcccgatacttatgggttttccgggggagctttgagagcatgttgtggcggcggaggtccgtacaacttcaacacctccgcacaatgcggtaacgagggagcaaccgcctgtgacaacccttcctcgtacgttaactgggacggttatcacttaacggaggctgcctatcaatgtattaccaccggtttgttagagggtccgtatacctatcctcgtatgaaaaatttgtgtcgctttgactctcgcaaaacatctcgagttgcccaaatttaa

SEQ ID NO: 3 - protein, 16 aa, Artificial
  Fragment 1 de IbGDSL
AVFGFGDSLTDAGNLI

SEQ ID NO: 4 - protein, 16 aa, Artificial
  Fragment 2 de IbGDSL
KPTGRCSDGRLIVDLI

SEQ ID NO: 5 - protein, 12 aa, Artificial
  Fragment 3 IbGDSL
FVLGEFGGNDYT

SEQ ID NO: 6 - protein, 20 aa, Artificial
  Fragment 4 IbGDSL
GATACDNPSSYVNWDGYHLT

SEQ ID NO: 7 - protein, 4 aa, Artificial
  Fragment 5 IbGDSL
GDSL

(19) United States
(12) Patent Application Publication
(10) Pub. No.: US 2023/0148181 A1
(43) Pub. Date: May 11, 2023

(54) ENZYME FOR THE CONVERSION OF CHLOROGENIC ACID INTO ISOCHLOROGENIC ACID

(71) Applicants: UNIVERSITE DE LORRAINE, NANCY (FR); UNIVERSITE DE LILLE, LILLE (FR); CELLENGO, VANDOEUVRE-LÈS-NANCY (FR)

(72) Inventors: Frédéric Jean BOURGAUD, VANDOEUVRE-LÈS-NANCY (FR); Sissi MIGUEL, MECLEUVES (FR); Aleksander SALWINSKI, NANCY (FR); Cindy SIGNE, LAXOU (FR); Alain HEHN, NEUVES MAISONS (FR); Jean-Louis HILBERT, VILLENEUVE D'ASCQ (FR); David GAGNEUL, LAMBERSART (FR); Guillaume LEGRAND, CAPPELLE-EN-PEVELE (FR)

(73) Assignees: UNIVERSITE DE LORRAINE, NANCY (FR); UNIVERSITE DE LILLE, LILLE (FR); CELLENGO, VANDOEUVRE-LÈS-NANCY (FR)

(21) Appl. No.: 17/910,937

(22) PCT Filed: Mar. 12, 2021
(86) PCT No.: PCT/EP2021/056403

§ 371 Date: Sep. 12, 2022

(30) Foreign Application Priority Data
Mar. 13, 2020 (FR) 2002518

(51) Int. Cl. C12P 7/62; C12N 9/18; C12N 15/81; C12N 15/82

(52) U.S. Cl. CPC C12P 7/62; C12N 9/18; C12N 15/815; C12N 15/8243

(57) ABSTRACT

A protein capable of converting chlorogenic acid into isochlorogenic acid. The protein includes or is an amino acid sequence SEQ ID No. 1, a sequence having at least 80% identity with this sequence or a fragment of this sequence. Also, a process for producing isochlorogenic acid from chlorogenic acid, which includes producing the protein and bringing it into contact with chlorogenic acid.

### FIELD

One object of the present invention is a protein whose enzymatic activity makes it possible to convert chlorogenic acid into isochlorogenic acid. Another object of the present invention is also a method for converting chlorogenic acid into isochlorogenic acid, comprising producing a protein according to the invention and its use for the production of isochlorogenic acid from chlorogenic acid. The invention is thus in the field of the production and use of a recombinant enzyme for the synthesis of a substance.

### BACKGROUND

Isochlorogenic acid, or 3,5-DiCaffeoylQuinic acid or 3,5-DCQ, is currently the subject of numerous studies and several pharmacological properties point to important developments especially in the field of prevention and/or treatment of diseases, such as Alzheimer's disease and cancer, viral diseases and in cosmetic applications. 3,5-DCQ can be purified from plants, especially from the sweet potato (Ipomoea batatas) (Harrison et al, “Contents of caffeoylquinic acid compounds in the storage roots of sixteen sweet potato genotypes and their potential biological activity”; J. Amer. Soc. Hort. Sci., 133(4):492-500, 2008). However, 3,5-DCQ is synthesised in relatively small amounts, so its purification from plants is laborious and expensive, and is therefore not compatible with widespread use. Furthermore, different caffeoylquinic acid isomers can be produced by the same plant cell, which further implies an additional step of isolating 3,5-DCQ compared to the other isomers.

There is therefore a need for a means for obtaining isochlorogenic acid in significant amounts in a reliable, easy and inexpensive manner.

The international application published as WO 2013/178 705 describes enzymes for converting chlorogenic acid (CGA) into di-, tri- or tetracaffeoylquinic acids. The function of said enzymes is close to that of HCT (Hydroxycynnamoyl-CoA shikimate/quinate hydroxycinnamoyl transferases) or HQT (Hydroxycynnamoyl-CoA quinate hydroxycinnamoyl transferases) proteins, belonging more generally to the family of BAHD acyltransferases.

Kojima and Kondo (“An enzyme in sweet potato root which catalyses the conversion of chlorogenic acid, 3-caffeoylquinic acid, to isochlorogenic acid, 3,5-dicaffeoylquinic acid”, Agric. Biol. Chem. 49(8), 2467-9, 1985) describe an enzyme, whose function is not precisely identified and whose structure is not disclosed, present in sweet potato extract and capable of converting chlorogenic acid into isochlorogenic acid.

Villegas et al (“Purification and characterization of chlorogenic potato roots”, Phytochemistry, vol. 26(6), 1577-81, 1987) describe a chlorogenate caffeoyltransferase capable of converting chlorogenic acid into isochlorogenic acid.)

Teutschbein et al (“Identification and localization of a lipase-like acyltransferase in phenylpropanoid metabolism of tomato (Solanum lycopersicum”, J. Biol. Chem, 285(49), pp. 38374-81, 2010) describe the identification of a chlorogenate glucarate caffeoyltransferase (CGT) in tomato extract.

### SUMMARY

The inventors have now isolated and cloned, from extracts of Ipomoea batatas, an enzyme belonging to the family of GDSL lipases/esterases, which are characterised by the presence within their amino acid sequence of the linkage of the following four amino acids: glycine (G)-aspartic acid (D)-serine (S)-leucine (L). This enzyme is referred to as: IbGDSL, for “GDSL enzyme of Ipomoea batatas. The inventors have also shown that this enzyme is capable of converting chlorogenic acid into isochlorogenic acid, with high substrate specificity and exclusive or near-exclusive production of 3,5-DCQ.

The present invention meets the above requirements, as after transformation of host cells transformed by a recombinant vector comprising a nucleotide sequence encoding a GDSL esterase/lipase enzyme according to the invention, and placed under appropriate culture conditions, the enzyme is detected in a total protein extract of said host cells. Furthermore, the inventors have demonstrated that said enzyme according to the invention is functional when present in an isolated form or in the culture medium of said host cell. The GDSL esterase/lipase according to the invention exclusively catalyses the formation of 3,5-DCQ, to the exclusion of any other isomer. Finally, the inventors have shown that the GDSL esterase/lipase according to the invention efficiently catalyses the conversion of chlorogenic acid present in a plant extract into 3,5-DCQ.

A first object of the present invention is therefore a protein capable of converting chlorogenic acid into isochlorogenic acid and comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1. A second object of the invention is a method for producing isochlorogenic acid (3,5-DCQ) from chlorogenic acid, this method implementing a protein according to the invention. A third object of the invention is the use of a protein according to the invention for the production of isochlorogenic acid (3,5-DCQ) from chlorogenic acid.

### BRIEF DESCRIPTION OF THE FIGURES

Further advantages and characteristics will become apparent from the detailed description of one embodiment which is not limiting, and from the appended figures, in which:

FIG. 1 (Example 1) represents the scheme of the enzymatic reaction conducted by IbGDSL, which catalyses the formation of 3,5-DCQ and quinic acid (QA) by the condensation of two CGA molecules.

FIGS. 2A and 2B (Example 1) respectively represent the detection of the enzyme by a western-blot assay performed using antibodies specifically directed against the six-histidine tag at the C-terminal position of the protein, which demonstrates the production of IbGDSL (predicted size 40.1 kDa) by N. benthamiana plants (FIG. 1A) and P. pastoris cells (FIG. 1B). In each figure, column 1 represents the analysis conducted on the production host transformed with the empty vector (negative control) and column 2 represents the analysis conducted on the production host transformed with the vector including the gene of interest.

FIGS. 3A, 3B and 3C (Example 2) respectively represent the 3,5-DCQ concentration (in mM) (FIGS. 3A and 3B), or the speed of bioconversion to 3,5-DCQ (in micromoles/minutes) (FIG. 3C), as a function of pH (FIG. 3A), temperature (FIG. 3B) or CGA substrate concentration (in mM) (FIG. 3C).

FIGS. 4A, 4B and 4C (Example 3) respectively represent chromatograms of the 3,5-DCQ standard (FIG. 4A) and the enzymatic reactions for the conversion of CGA into 3,5-DCQ carried out with the supernatants of P. pastoris transformed with the empty vector (FIG. 4B) or transformed with the vector carrying the gene encoding IbGDSL (FIG. 4C) after 3 days of methanol induction. Peaks with retention times of 3.2 minutes and 8.3 minutes correspond to CGA (m/z neg 353) and 3,5-DCQ (m/z neg 515) respectively.

FIGS. 5A and 5B (Example 3) represent chromatograms of the bioconversion reactions of chlorogenic acid to 3,5-DCQ via the addition of the substrate directly into P. pastoris cultures transformed with either the empty vector (FIG. 5A) or the vector carrying the gene encoding IbGDSL (FIG. 5B). In each figure, from top to bottom and shifted to the right, are the chromatograms corresponding to T0 before CGA addition, 6 hours after CGA addition and 120 hours after CGA addition, respectively. The peaks with retention times of 3.2 minutes and 8.3 minutes correspond to CGA (m/z neg 353) and 3,5-DCQ (m/z neg 515) respectively.

FIGS. 6A and 6B (Example 3) respectively represent the conversion rate of pure CGA to 3,5-DCQ (in mole %) (FIG. 6A) or the 3,5-DCQ concentration (in mg/L) (FIG. 6B), as a function of the reaction time (in hours). The theoretical limit at 50% corresponds to the maximum yield obtained when all CGA molecules are transformed into 3,5-DCQ (stoichiometric yield of 2:1 respectively). FIG. 6A: curves with triangles (5 mM or 1.9 g/L CGA), light crosses (7.5 mM or 2.6 g/L CGA), dark crosses (9 mM or 3.2 g/L CGA), dark squares (10 mM or 3.6 g/L CGA), light squares (15 mM or 5.5 g/L CGA). FIG. 6B: Curves with triangles (5 mM or 1.9 g/L CGA), light crosses (7.5 mM or 2.6 g/L CGA), lines (9 mM or 3.2 g/L CGA), dark squares (10 mM or 3.6 g/L CGA), light squares (15 mM or 5.5 g/L CGA).

FIG. 7A (Example 4) represents a chromatogram showing the composition of caffeic acid derivatives in a green coffee hydroalcoholic extract, with: peak 1: MCQ1 (other mono-caffeoylquinic acid isomer), peak 2: MCQ2 (other mono-caffeoylquinic acid isomer), peak 3: CGA (chlorogenic acid, 5-O-caffeoyl quinic acid); peak 4: CA (caffeic acid); peak 5: MFQ (monoferuloyl quinic acid); peak 7: 4,5-DCQ (3,4 dicaffeoyl quinic acid); peak 8: 3,5-DCQ (3,5 dicaffeoyl quinic acid); peak 9: 3,4-DCQ (4,5 dicaffeoyl quinic acid).

FIG. 7B (Example 4) represents a chromatogram of a green coffee hydroalcoholic extract obtained before (black trace) and after 50 h (light trace) of bioconversion by IbGDSL, in green coffee extract; the initial substrate concentration is equivalent to 10 mM CGA. Peak 1: MCQ; peak 2: CGA; peak 3: MFQ; peak 4: 4.5-DCQ; peak 5: 3,5-DCQ; peak 6: 3,4-DCQ. The difference between the light and dark traces is especially visible in peak 5: 3,5-DCQ.

FIG. 8 (Example 4) represents the DCQ content (mg/L) obtained for different concentrations of green coffee extract, expressed as CGA equivalent, as a function of the reaction time (in hours). The supernatant of P. pastoris culture medium containing the GDSL enzyme was concentrated 37-fold. Curve with solid line (CGA 1 mM), light circles (CGA 3.5 mM), diamonds (CGA 5 mM), dark squares (CGA 10 mM).

FIG. 9 (Example 4) represents the 3,5-DCQ content (mg/L) measured in a green coffee extract bioconverted by a cell suspension of P. pastoris expressing IbGDSL (GDSL+) or not expressing IbGDSL (GDSL−), as a function of time (in days).

FIG. 10 (Example 4) represents the 3,5-DCQ content (mg/L) obtained by enzymatic bioconversion by IbGDSL of a green coffee solution containing 5 mM CGA, as a function of time (in hours) when the supernatant of P. pastoris culture medium containing the GDSL enzyme was concentrated 10-fold (circles), 20-fold (triangles) or 37-fold (squares).

### DETAILED DESCRIPTION

According to a first object, the invention relates to a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid.

According to one particular aspect of this first object, the invention relates to a recombinant protein, that is a protein produced by a cell whose genetic material has been modified.

By “comprising” it is meant that the element is present but that other elements may also be present. In the case of an amino acid sequence, the sequence in question may especially further comprise additional amino acids, on the N-terminal or C-terminal side of said sequence, these additional amino acids making it possible especially to facilitate the characterisation and/or purification of the protein of interest. In the case of a nucleotide sequence, the sequence in question may especially further comprise additional nucleotides on the 3′ or 5′ side of said sequence.

By “consisting of”, it is meant that no other elements than those mentioned are present. However, this limit includes possible post-translational modifications of the protein of interest.

By “having at least 80% identity”, it is meant that said sequences have at least 80% identity after optimal overall alignment, that is by overall alignment between two sequences giving the highest percentage of identity between them. The optimal global alignment of two sequences can especially be carried out according to the Needleman-Wunsch algorithm, well known to the person skilled in the art (Needleman & Wunsch, “A general method applicable to the search for similarities in the amino acid sequences of two proteins”, J. Mol. Biol, 48(3):443-53). The proteins according to the invention comprise, or consist of, an amino acid sequence having at least 80%, advantageously at least 81%, 82%, 83%, 84%, 85%, 86%, 87%, 88%, 89%, 90%, 91%, 92%, 93%, 94%, 95%, 96%, 97%, 98% or 99% identity with the amino acid sequence SEQ ID No. 1 after optimal overall alignment. Advantageously, the proteins according to the invention comprise, or consist of, an amino acid sequence having at least 99.1%, 99.2%, 99.3%, 99.4%, 99.5%, 99.6%, 99.7%, 99.8% or 99.9% identity with the amino acid sequence SEQ ID No. 1 after optimal global alignment.

According to one particular aspect, among the proteins according to the invention comprising, or consisting of, an amino acid sequence having at least 80% identity with the amino acid sequence SEQ ID No. 1 and capable of converting chlorogenic acid into isochlorogenic acid, the proteins comprising at least one sequence selected from: SEQ ID No. 3, SEQ ID No. 4, SEQ ID No. 5 and SEQ ID No. 6 are preferred.

According to another particular aspect, among the proteins according to the invention comprising, or consisting of, an amino acid sequence having at least 80% identity with the amino acid sequence SEQ ID No. 1 and capable of converting chlorogenic acid into isochlorogenic acid, the proteins comprising:
a serine amino acid at position 11
and/or an aspartic acid amino acid at position 317
and/or an aspartic acid amino acid at position 153
and/or a histidine amino acid at position 320 are preferred.

According to an even more particular aspect, among the proteins according to the invention comprising, or consisting of, an amino acid sequence having at least 80% identity with the amino acid sequence SEQ ID No. 1 and capable of converting chlorogenic acid into isochlorogenic acid, proteins comprising a serine amino acid at position 11, an aspartic acid amino acid at position 317, an aspartic acid amino acid at position 153 and a histidine amino acid at position 320 are preferred.

According to another particular aspect, among the proteins according to the invention comprising, or consisting of, an amino acid sequence fragment having at least 80% identity with the amino acid sequence SEQ ID No. 1 and capable of converting chlorogenic acid into isochlorogenic acid, proteins comprising a serine amino acid at position 11 and/or an aspartic acid amino acid at position 317 and/or an aspartic acid amino acid at position 153 and/or a histidine amino acid at position 320 are preferred.

According to this aspect, the proteins according to the invention comprising, or consisting of, an amino acid sequence fragment having at least 80% identity with the amino acid sequence SEQ ID No. 1 and capable of converting chlorogenic acid into isochlorogenic acid include at least 50 amino acids, preferably at least 100 amino acids and more preferably at least 150 amino acids.

By “chlorogenic acid”, it is meant the simple ester of caffeic acid and quinic acid, also referred to as caffeoylquinic acid or trans-5-O-caffeoyl-D-quinate, of the following formula (I)

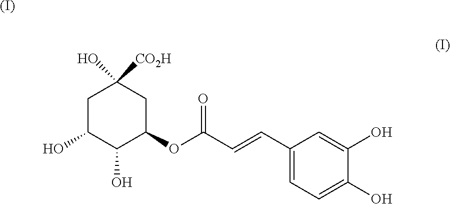 [SMILES: "(I)" ; O=C(/C=C/c1ccc(O)c(O)c1)O[C@@H]1C[C@](O)(C(=O)O)C[C@@H](O)[C@H]1O]

A dicaffeoylquinic acid, abbreviated as DCQ, is a diester comprised of a quinic acid molecule in which two of the four alcohol functions have been esterified with a caffeic acid molecule. The general formula of QCDs is as follows (II), in which R₂, R₃, R₄ and R₅ each independently represent a caffeoyl group or a hydrogen atom, with the proviso that at least two of R₂, R₃, R₄ and R₅ are different from a hydrogen atom:

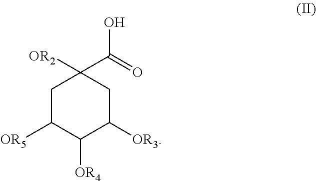

By “isochlorogenic acid”, also referred to as 3,5 dicaffeoylquinic acid or “3,5-DCQ”, it is meant a di-ester comprised of a quinic acid molecule whose alcohol functions 3 and 5 are esterified with a caffeic acid molecule, according to the following formula (III). 3,5-DCQ is naturally present especially in an extract of sweet potato, Ipomoea batatas.

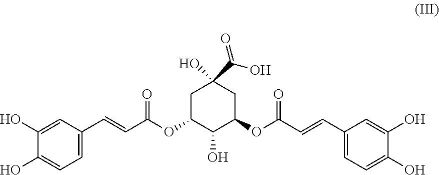 [SMILES: O=C(/C=C/c1ccc(O)c(O)c1)O[C@@H]1C[C@](O)(C(=O)O)C[C@@H](OC(=O)/C=C/c2ccc(O)c(O)c2)[C@H]1O]

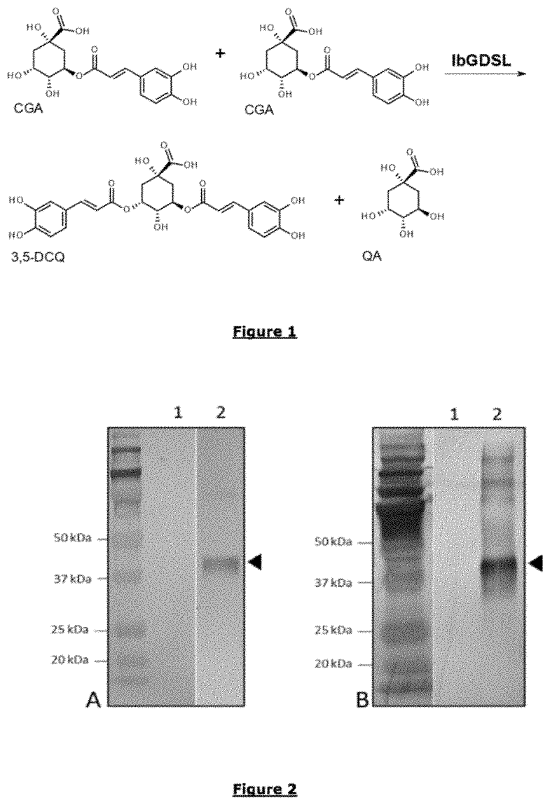

By “capable of converting chlorogenic acid into isochlorogenic acid”, it is meant a protein which, when placed under adapted reaction conditions, is capable of catalysing the formation of chlorogenic acid to isochlorogenic acid by the condensation of two chlorogenic acid molecules, according to the reaction scheme described in FIG. 1.

More particularly, a protein according to the invention is capable of converting chlorogenic acid into isochlorogenic acid predominantly, and preferably exclusively or almost exclusively.

Even more particularly, the catalytic activity as defined above of a protein according to the invention meets one, two, three or four of the following characteristics: a) Vmax (maximum initial speed) of between 60 and 240 nanomoles·s⁻¹), b) Km (Michaëlis constant) of between 2 and 5 mM (with respect to CGA), c) optimal operating pH of between 6 and 6.6; d) optimal operating temperature of between 39 and 41° C.

In particular, the catalytic activity as defined above of a protein according to the invention meets one, two, three or four of the following characteristics: a) Vmax (maximum initial speed) of 7.18 micromol·min⁻¹ (that is 120 nanomoles·s⁻¹); b) Km (Michaëlis constant) of 3.5 mM (with respect to CGA); c) optimal operating pH of 6.3; d) optimal operating temperature of 39.9° C.

According to a more particular aspect, the invention relates to a protein comprising, or consisting of, an amino acid sequence selected from: SEQ ID No. 1, a sequence having at least 95% identity with SEQ ID No. 1 and comprising the sequence SEQ ID No. 7, said protein being capable of converting chlorogenic acid into isochlorogenic acid.

More particularly, the invention relates to a protein comprising, or consisting of, an amino acid sequence having at least 80% identity with the amino acid sequence SEQ ID No. 1 and capable of converting chlorogenic acid into isochlorogenic acid, said protein being selected from the so-called “GDSL esterase/lipase” enzymes, and in particular from the “GDSL esterase/lipase” enzymes of Ipomoea, more particularly from the “GDSL esterase/lipase” enzymes of Ipomoea batatas, and from the “GDSL esterase/lipase” enzymes of Solanaceae, Asteraceae and Rubiaceae.

Still more particularly, the invention relates to a protein comprising, or consisting of, the selected amino acid sequence SEQ ID No. 1, said protein being capable of converting chlorogenic acid into isochlorogenic acid and being selected from the GDSL esterase/lipase enzymes of Ipomoea batatas.

According to another particular aspect, one object of the invention is an isolated nucleic acid molecule encoding a protein according to the invention.

More particularly, one object of the invention is an isolated nucleic acid molecule encoding a protein according to the invention, said molecule comprising, or being constituted by, a nucleic acid sequence selected from: SEQ ID No. 2 and a sequence having at least 80% identity with SEQ ID No. 2. Due to the degeneracy of the genetic code, different nucleic acid sequences can encode the proteins according to the invention. Depending on the host selected to produce a protein according to the invention, the degeneracy of the nucleic code can be used to adapt the codons of the nucleotide sequence to the codon usage preferably found in the selected host, so as to optimise the expression of the protein of interest in the host protein. A person skilled in the art wishing to produce a recombinant protein in one particular host cell, such as for example a yeast cell or a plant cell, will have easy access to the optimal codons, by virtue of readily available codon optimization software. More particularly, one object of the invention is an isolated nucleic acid molecule encoding a protein according to the invention, said molecule comprising or consisting of a nucleic acid sequence selected from: SEQ ID No. 2 and a sequence having at least 95% identity with SEQ ID No. 2.

According to an even more particular aspect, one object of the invention is a recombinant vector comprising at least one nucleic acid molecule according to the invention, each of said at least one molecule being placed under the control of the means necessary for the expression of said protein in a given host cell. Such a vector may especially be selected from plasmids, yeast artificial chromosomes (YACs), binary type vectors (pBIN, pGW) and any type of appropriate vector as a function of the host cell selected. Said means necessary for the expression of said protein in a host cell are well known to the person skilled in the art. By “means necessary for the expression of said protein in a host cell”, it is meant any means enabling said protein to be obtained, especially a promoter, a transcription terminator, an origin of replication, and possibly a selection marker, said means being operatively linked to the nucleotide sequence encoding the protein of interest. A vector according to the invention may further comprise a nucleotide sequence encoding a means ensuring the export of the protein produced into the culture medium of the host cell and/or a nucleotide sequence encoding a means for enabling the purification of the protein produced. Such means are well known to the person skilled in the art, who can therefore easily select them and insert, in a functional manner, said nucleotide sequences. To ensure the purification of said protein, one of the known means consists of a histidine tag, or series of histidine amino acids.

More particularly, one object of the invention is a recombinant vector comprising at least one nucleic acid molecule according to the invention, each of said at least one molecule being placed under the control of means necessary for the expression of said protein in a yeast host cell or under the control of means necessary for the expression of said protein in a plant host cell.

The means necessary for the expression of said protein in a yeast host cell are well known to the person skilled in the art, they are especially present in the pPICZa, pPIC9K, pAOX815 vectors marketed by the ThermoFischer company.

The means necessary for the expression of said protein in a plant host cell, and especially in a plant cell commonly used for the production of exogenous proteins, such as tobacco, rice, tomato or carnivorous plant cells, are also well known to the person skilled in the art.

According to another particular aspect, one object of the invention is a host cell comprising at least one isolated nucleic acid molecule according to the invention or at least one recombinant vector comprising at least one nucleic acid molecule according to the invention, each of said at least one molecule being placed under the control of means necessary for the expression of said protein in a host cell.

More particularly, the invention relates to a host cell selected from yeast cells, in particular yeast cells of the Pichia pastoris, Saccharomyces cerevisiae, Yarrowia lipolytica, Komagataella sp. and Kluyveromyces lactis, and preferably Pichia pastoris strain.

According to another particular aspect, the invention relates to a host cell selected from plant cells, in particular Nicotiana benthamiana Ipomoea batatas, Nicotiana tabacum, Arabidopsis thaliana, Zea mays, rice, Coffea arabica, tomato, Asteraceae (thistles, artichokes) etc.

According to another particular aspect, the invention relates to a transgenic plant comprising at least one isolated nucleic acid molecule according to the invention, at least one recombinant vector comprising at least one nucleic acid molecule according to the invention, or at least one plant host cell according to the invention. Preferably, the invention relates to a transgenic plant comprising at least one isolated nucleic acid molecule according to the invention, at least one recombinant vector comprising at least one nucleic acid molecule according to the invention, or at least one Nicotiana benthamiana Ipomoea batatas, Nicotiana tabacum, Arabidopsis thaliana, Zea mays, rice, Coffea arabica, tomato or Asteraceae host cell according to the invention.

According to a second object, the invention relates to a method for producing isochlorogenic acid comprising contacting, under appropriate reaction conditions, chlorogenic acid and a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to obtain an isochlorogenic acid-enriched composition.

According to one particular aspect, the invention relates to a method for producing isochlorogenic acid comprising contacting, under appropriate reaction conditions, chlorogenic acid and a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to obtain an isochlorogenic acid-enriched composition, said method further comprising:
a prior step of culturing, in an appropriate culture medium and under appropriate conditions, a host cell capable of expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein produced being optionally purified, and/or said chlorogenic acid is present in isolated form or in a composition, said composition being especially a plant extract, and/or
a subsequent step of isolating the isochlorogenic acid produced during the reaction.

According to one particular aspect, the invention relates to a method wherein said protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, is purified, prior to contacting, with chlorogenic acid. The purification is carried out by any means known to the person skilled in the art.

According to another particular aspect, the invention relates to a method wherein, when contacting said protein with chlorogenic acid, said protein is present in a composition or mixture, especially the culture medium, or supernatant, of a recombinant host cell which has produced said protein.

More particularly, according to this second object, the invention relates to a method for producing isochlorogenic acid further comprising a prior step of culturing, in an appropriate culture medium and under appropriate conditions, a host cell capable of expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1.

By “appropriate reaction conditions”, it is meant the various parameters enabling the enzymatic reaction conducted by IbGDSL to be carried out, which catalyses the formation of 3,5-DCQ and quinic acid (QA) by the condensation of two CGA molecules. The duration of the reaction is preferably more than 4 hours, preferably between 4 and 60 hours, preferably 50 hours. The pH of the reaction is between 5 and 7, preferably 6±0.5. The temperature of the reaction is between 30 and 40° C., and is preferably 35° C.±5° C. The concentration of GDSL per volume of plant extract is between 0.5 mg/L to 10 mg/L, preferably 4.2 mg GDSL/L±3.4.

According to another particular aspect of this second object, the invention relates to a method for producing isochlorogenic acid further comprising a subsequent step of isolating the isochlorogenic acid produced during the reaction.

According to a more particular aspect of this second object, the invention relates to a method for producing isochlorogenic acid comprising the steps of:
i) culturing, in an appropriate culture medium and under appropriate conditions, a host cell capable of expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, to express a protein capable of converting chlorogenic acid into isochlorogenic acid,
ii) contacting, under appropriate reaction conditions, the protein expressed in step i) with chlorogenic acid, and
iii) isolating the isochlorogenic acid produced during the reaction of step ii).

In a method according to the second object of the invention, said chlorogenic acid is present either in isolated form or in a composition, said composition being especially a plant extract. By “plant extract”, it is meant the result of the extraction of the active principles of a plant, or of at least part of a plant, by fermentation, maceration, decoction or infusion. Said plant extract may especially be added in the form of a liquid or a powder. Preferably, in such a method according to the invention, a plant extract comprises at least 5% CGA. Such a plant extract may be selected from plant extracts of coffee, blueberry, sunflower, great burdock, chicory, artichoke, Japanese medlar, prune, mint, carrot, potato, apple and pear.

The invention further relates to a method for producing isochlorogenic acid comprising the steps of:
i) culturing, in an appropriate culture medium and under appropriate conditions, a host cell capable of expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, to express a protein capable of converting chlorogenic acid into isochlorogenic acid,
ii) contacting, under appropriate reaction conditions, the protein expressed in step i) with chlorogenic acid, and
iii) isolating the isochlorogenic acid produced during the reaction of step ii).

According to a particular aspect, a method for producing isochlorogenic acid according to the invention comprises the steps of:
i) culturing, in an appropriate culture medium and under appropriate conditions, a Pichia pastoris host cell capable of expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, to express a protein capable of converting chlorogenic acid into isochlorogenic acid, said protein being produced and then exported in the culture supernatant of P. pastoris cells,
ii) contacting, under appropriate reaction conditions, the protein expressed in step i) with chlorogenic acid present in a green coffee extract, and
iii) isolating the isochlorogenic acid produced during the reaction of step ii).

According to another particular aspect, a method for producing isochlorogenic acid according to the invention comprises the steps of:
contacting, under appropriate reaction conditions, a protein capable of converting chlorogenic acid into isochlorogenic acid and comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with
SEQ ID No. 1, with chlorogenic acid present in a green coffee extract, and isolating the isochlorogenic acid produced during the reaction of the preceding reaction.

By “green coffee”, it is meant the beans of plants of the Coffea genus before cooking or roasting, especially the beans of the Coffea canephora or Coffea arabica species. Preferably, by “green coffee”, it is meant Coffea canephora.

By “green coffee extract” it is meant an extract obtained by solid/liquid extraction in ethanol to recover the metabolites contained in dried green coffee beans. The ethanol used is pure or in the form of an aqueous alcohol solution, the latter comprising from 10% to 99.9% alcohol, more particularly between 40% and 90%, and even more particularly between 50% and 85%. Optionally, the caffeine is removed from the extract by treatment with ethyl acetate or an ethyl acetate/hexane mixture. The extract is reduced to powder form by implementing any adapted method known to the person skilled in the art, especially atomisation or freeze-drying.

In a green coffee extract according to the invention, chlorogenic acid is present at a minimum concentration of at least 2 mM, preferably at least 5 mM, at least 7.5 mM, preferably 10 mM.

According to an even more particular aspect, a method for producing isochlorogenic acid according to the invention comprises the steps of:
culturing, in an appropriate culture medium and under appropriate conditions, a Pichia pastoris host cell capable of expressing a protein comprising, or consisting of, an amino acid sequence selected from: SEQ ID No. 1, to express a protein capable of converting chlorogenic acid into isochlorogenic acid, said protein being produced and then exported in the culture supernatant of P. pastoris cells
contacting, under appropriate reaction conditions, the protein expressed in step i) with chlorogenic acid present in a green coffee extract, and
isolating the isochlorogenic acid produced during the reaction of the previous step.

According to another particular aspect, one object of the invention is the product likely to be obtained by a method according to the invention, said method comprising contacting, under appropriate reaction conditions, a green coffee extract whose chlorogenic acid concentration is greater than or equal to 2 mM, and a protein capable of converting chlorogenic acid into isochlorogenic acid and comprising, or consisting of, an amino acid sequence selected from:
SEQ ID NO. 1,
A sequence having at least 80% identity with SEQ ID No. 1,
A fragment including at least 50 amino acids of said SEQ ID No. 1

and
a fragment including at least 50 amino acids of said sequence having at least 80% identity with SEQ ID No. 1.

According to another more particular aspect, one object of the invention is the product likely to be obtained by a method according to the invention, said method comprising:
A step of culturing P. pastoris transformed by an expression vector having integrated the gene encoding the IbGSDL protein of SEQ ID No. 1, in a, appropriate nutrient medium and under appropriate conditions, said protein being produced and then exported in the culture supernatant,
Optionally purifying the protein of SEQ ID No. 1,
Contacting, under appropriate reaction conditions, a green coffee extract whose chlorogenic acid concentration is greater than or equal to 2 mM, with said culture supernatant, or optionally with said purified protein.
Optionally a subsequent step of isolating the isochlorogenic acid produced during the reaction.

According to a third object, the invention relates to the use of at least one protein, said protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, or of a host cell expressing such a protein, to convert chlorogenic acid into isochlorogenic acid.

More particularly, the invention relates to the use of at least one protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to convert chlorogenic acid into isochlorogenic acid, said protein being contacted with chlorogenic acid, under appropriate reaction conditions, without having been previously isolated from the host cell or from the culture medium in which the host cell was cultured to produce said protein.

According to another particular aspect, the invention relates to the use of at least one protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to convert chlorogenic acid into isochlorogenic acid, said protein being contacted with chlorogenic acid, under appropriate reaction conditions, after having been previously isolated or purified from the host cell or from the culture medium in which the host cell was cultured to produce said protein. Even more particularly, in a use according to the invention, said protein, isolated from the host cell or purified from the culture medium, is added to a solution comprising predominantly, or only, chlorogenic acid. Alternatively, in a use according to the invention said protein, isolated from the host cell or purified from the culture medium, is added to an extract comprising especially chlorogenic acid.

According to another particular aspect of this third object, the invention relates to the use of at least one host cell expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to convert chlorogenic acid into isochlorogenic acid.

More particularly, one object of the invention is the use of at least one yeast cell expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to convert chlorogenic acid into isochlorogenic acid. Preferably, said yeast cell is a cell of the Pichia pastoris strain transformed by a recombinant vector comprising at least one nucleic acid molecule according to the invention, each of said at least one molecule being placed under the control of means necessary for the expression of said protein in a host cell. Such a vector is especially selected from plasmids, yeast artificial chromosomes (YACs), binary type vectors (pBIN, pGW) and any type of appropriate vector as a function of the host cell.

According to another particular aspect of this third object, the invention relates to the use of at least one plant host cell expressing a protein comprising, or consisting of, an amino acid sequence selected from: SEQ ID No. 1, a sequence having at least 80% identity with SEQ ID No. 1, a fragment of said SEQ ID No. 1 and a fragment of said sequence having at least 80% identity with SEQ ID No. 1, said protein being capable of converting chlorogenic acid into isochlorogenic acid, to convert chlorogenic acid into isochlorogenic acid.

The present invention will be better understood from the following examples 1 to 4, which are given to illustrate the invention and not to limit its scope.

### EXAMPLES

### Example 1: Identification, Production and Characterisation of a Recombinant GDSL Enzyme of Ipomoea batatas

### 1.1 Materials and Methods

A complementary DNA library of I. batatas was prepared according to the protocol described in document W02013/178 705 from roots of plants cultivated under 3,5-DCQ-rich aeroponic conditions. In parallel, fragmentation of a complex protein extract from a 3,5-DCQ-rich I. batatas tuber was performed in order to select, step by step, proteins with isochlorogenic acid synthase activity. At the end of this experiment, SDS-PAGE profiles were obtained and the observed proteins were sequenced. 400 peptides were obtained and their amino acid sequence was aligned with the I. batatas cDNA library using the tBlastn program which compares a peptide sequence to the translation products of a nucleotide sequence. Thus, sequences with high homologies to the sequenced peptides were detected among all sequences composing the tissue transcriptome.

After identifying a candidate sequence from the cDNA library, its coding sequence was isolated from an extraction of total messenger RNAs converted into cDNA. For this, mRNAs from I. batatas roots cultivated under aeroponic conditions were extracted using a commercial extraction kit specific to plant tissues according to the supplier's instructions (RNeasy® Plant Mini Kit-QIAGEN). After assaying and checking the quality of the extracted mRNAs, the cDNA encoding the candidate protein was amplified using, on the one hand, sequence-specific primers designed to add a six-histidine tag at the C-terminal position of the protein, necessary for its detection after production and for its purification, and, on the other hand, a commercial kit allowing the conversion of mRNA into cDNA and the amplification of the sequence by PCR (Polymerase Chain Reaction) (SuperScript™ III One-Step RT-PCR System with Platinum™ Taq High Fidelity DNA Polymerase—INVITROGEN) in a single step. The obtained amplicon was then cloned into a basic commercial vector (pCR™8/GW/TOPO™—INVITROGEN) allowing its sequencing and integration into several vectors dedicated to various expression systems.

Peptide alignment on the Ipomea batatas cDNA library allowed the identification of a gene encoding a GDSL esterase/lipase among all the sequenced transcripts. The peptide and nucleotide sequences encoding IbGDSL are SEQ ID No. 1 and SEQ ID No. 2 respectively.

The gene encoding IbGDSL is subsequently integrated into an expression vector dedicated to plant cells by homologous recombination. At the time of gene amplification, a tag comprised of 6 histidines is added at the end of the gene to obtain a C-terminal tagged protein after transcription and translation. This tag allows easy purification of the protein by affinity chromatography.

This vector is then introduced into Agrobacterium tumefaciens of EHA105 strain capable of transfecting a DNA of interest into plant cells according to the freeze-thaw method described in the work of Chen et al (“Enhanced recovery of transformants of Agrobacterium tumefaciens after freeze-thaw transformation and drug selection. BioTechniques 16 (4): 664-68, 1994). Bacteria that have integrated the vector will have acquired resistance to an antibiotic at the same time, thus allowing them to be selected from non-transformed bacteria in the presence of this antibiotic agent.

For the transient transformation of N. benthamiana, agrobacteria carrying the recombinant vector are cultured in 15 mL of nutrient medium supplemented with the selection antibiotic and incubated at 28° C. under agitation at 200 rpm for 24 hours. The next day, 3 hours before transformation, 100 pM acetosyringone is added to the agrobacterial cultures to activate their virulence. After this time, the bacteria were centrifuged and transferred to nutrient medium once or twice to remove the antibiotics and finally transferred to infiltration buffer at pH 5.6 (10 mM SS, 100 μM acetosyringone). The OD₆₀₀ₙₘ (optical density) of the bacterial suspension is adjusted to 0.5.

The aerial parts of several 3-4 week old N. benthamiana plants cultured in a culture chamber with a photoperiod of 16 h/8 h day/night under artificial light (70 μmol m−2 s−1) at 26° C. with 70% humidity are fully immersed in the agrobacterial solution and subjected to vacuum infiltration in a bell jar connected to a pump. A vacuum step is performed down to 20 mbar to induce entry of the agrobacteria into the tissue before recovery to atmospheric pressure conditions. The N. benthamiana plants were then returned to culture under the same environmental conditions described above for 6 days. It is during this time that the agrobacteria will transfect the DNA of interest corresponding to the IbGDSL gene into the plant cells and the protein will be produced by the transcription and translation machinery of the host cells.

Confirmation of protein production by this system was determined by western-blot using an antibody specifically directed against the 6-histidine tag added at the C-terminal position of the protein (FIG. 2A).

In parallel, the gene encoding the protein is integrated into an expression vector dedicated to P. pastoris by homologous recombination. This vector allows the expression and secretion of the protein in the culture medium in the presence of methanol. The conventional P. pastoris transformation protocol used in this work is described in Cregg and Russell (“Transformation”. In Pichia Protocols, published by David R. Higgins and James M. Cregg, 27-39. Methods in Molecular Biology™ Totowa, N.J.: Humana Press. https://doi.org/10.1385/0-89603-421-6:27, 1998). Confirmation of enzyme production was conducted as described above by western blot (FIG. 2B).

### 1.2 Results

The results show that, after transformation of the production hosts, the enzyme was detected by western blot in a total protein extract from agroinfiltrated N. benthamiana leaves and in the supernatant of genetically transformed yeasts. Thus, unlike the negative control (FIG. 2A well 1), a band around 40 kDa is observed for N. benthamiana tissues agroinfiltrated for 6 days with the gene encoding IbGDSL (FIG. 2A well 2). Similarly, the P. pastoris culture transformed with the IbGDSL gene produced and secreted the enzyme into the culture medium within 3 days (FIG. 2B well 2) unlike the negative control transformed with the empty vector (FIG. 2A well 1).

### Example 2: Characterisation of the Catalytic Activity of Purified Recombinant IbGDSL

The in vitro enzyme assays are conducted on the purified recombinant enzyme produced in a plant system. After 6 days of co-culture, N. benthamiana leaves agro-infiltrated with the vector carrying the gene of interest, are harvested and ground in extraction buffer (20 mM sodium phosphate, 0.5 M NaCl, pH 7.4). The extract is then centrifuged and the supernatant containing all the soluble proteins including IbGDSL is recovered and sterilised by 0.2 μm filtration. Protein purification is then conducted according to the supplier's instructions on Nickel columns (HisTrap HP-GE HEALTHCARE). The elution fraction recovered after purification containing the protein is concentrated and desalted on centrifugation units with a cut-off at 10 kDa (Amicon® Ultra 0.5 mL Centrifugal Filters-PMNL 10 kDa—MILLIPORE).

Enzyme assays are performed in a 50 μl volume with 200-400 ng of the purified protein (that is a few μl). The optimal reaction pH will be determined using a polybuffer (0.1 M Tris/20 mM MES/0.1 M acetic acid) from which a pH range of 4 to 9 will be constructed. A 100 mM chlorogenic acid (CGA) stock solution is prepared shortly before the experiments from a CGA powder with a purity level of over 99% diluted in the polybuffer at pH 6.5. After each reaction, 150 μl of absolute ethanol is added to the 50 μl reaction mixture to stop the reaction and extract the produced molecules present in the reaction. The assay was then centrifuged and the supernatant recovered for UPLC-MS analysis.

The apparatus used for the analysis step is a Shimadzu Nexera X2 UPLC (LC-30AD pumps, SIL-30AC autosampler, CTO-20A oven, SPD-M20A diode array detectors; Kyoto, Japan) operating in reverse phase with a Kinetex Biphenyl column (00F-4622-AN, Phenomenex, Torrance, Calif., USA) of dimensions 150 mm×2.1 mm, 2.6 μm. The mobile phase consisted of solvent A (Mili-Q ultrapure water, Merck Millipore+0.1% formic acid, Carlo Erba, Val-de-Reuil, France) and a solvent B (Acetonitrile, Sigma-Aldrich Chemie GmbH, Steinheim, Germany) whose gradient was programmed as follows: phase B (%) 5-25% (0-10 min); 25-90% (10-10.5 min); 90% (10.5-12 min), 90-5% (12-12.1 min), 5% (12.1-14.1 min). The analysis flow rate is 0.5 mL/min with an oven temperature of 40° C. At the column outlet, a diode array detector records the UV spectra between 220 and 370 nm. The instrument is coupled to a mass spectrometer (Shimadzu LCMS-2020) operating with electrospray ionisation (4.5 kV) in negative mode in the m/z range between 100 and 1,000. LabSolutions software (version 5.60 SP2) is used to operate the system.

3,5-DCQ quantification is done by measuring the area of the peak of a 3,5-DCQ standard at 330 nm. The 3,5-DCQ standard is prepared at a concentration of 100 mg/L in a 70/30 DMSO/water mixture and acidified to pH 3 with hydrochloric acid. The contents of the other compounds (chlorogenic acid and the other DCQ isomers) are expressed as 3,5-DCQ equivalents. The content is calculated according to the following formula for a compound:

$$3.5 - DCQ content in [ \frac{mg}{L} ] = 100 \cdot \frac{\begin{matrix}( area of the peak corresponding to \\ 3.5 - DCQ at 330 nm of the sample )\end{matrix}}{\begin{matrix}( area of the peak corresponding to 3.5 - DCQ \\ at 330 nm of the standard solution )\end{matrix}}$$

3,5-DCQ is used as the quantification standard for the different 3,4-DCQ and 4,5-DCQ compounds because they belong to the same family of molecules.

Experiments to determine the physicochemical parameters required for IbGDSL to convert CGA into 3,5-DCQ were conducted on the purified enzyme produced in the plant expression system.

IbGDSL is an esterase/lipase capable of condensing two CGA molecules into 3,5-DCQ by transferring the caffeoyl group from one CGA molecule to another CGA molecule (FIG. 1). Therefore, CGA is used here as an acyl donor. The addition of caffeic acid to the reaction medium does not promote the formation of 3,5-DCQ.

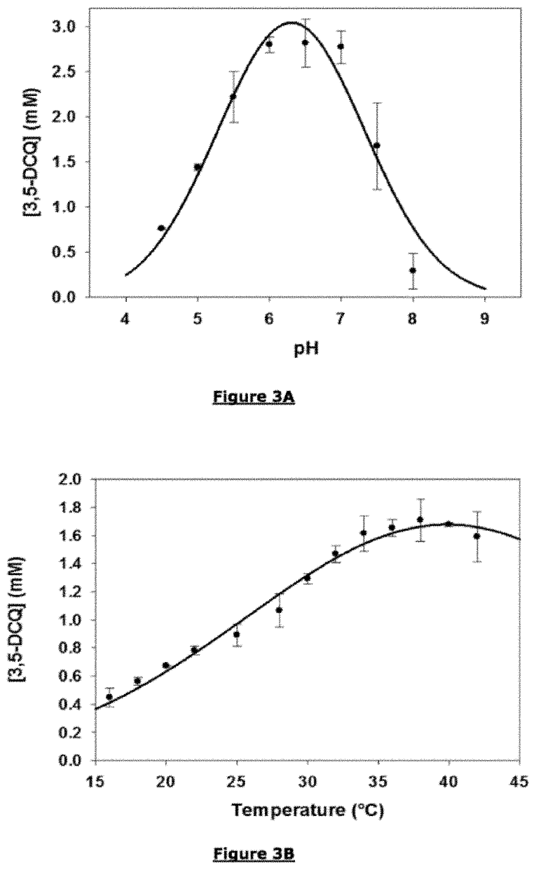

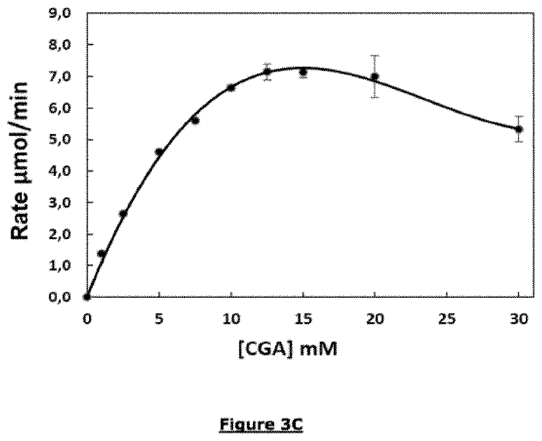

A pH range between 4 and 9 was established to determine the optimal pH for the conversion of CGA into 3,5-DCQ by virtue of IbGDSL. In a 50 μL reaction, a fixed amount of purified IbGDSL is contacted with a fixed concentration of 10 mM CGA at different pH conditions set by the polybuffer. The reaction was incubated for 30 minutes at 25° C. and stopped with ethanol. From the curve obtained it was determined that the optimal reaction pH is between 6 and 7 (FIG. 3A).

To determine the optimal reaction temperature for the conversion of CGA into 3,5-DCQ by virtue of IbGDSL, a temperature range of 15-45° C. was established. With a fixed amount of purified IbGDSL, a fixed concentration of 10 mM CGA and the polybuffer at pH 6.5, the optimal temperature for conversion is estimated to be between 34 and 41° C., with a maximum at 37° C. (FIG. 3B).

A CGA concentration range was established to determine the threshold substrate concentration at which inhibition of IbGDSL activity by the product is observed. With a fixed amount of enzyme and a pH set at 6.5, a slowing down of the enzyme activity is observed from 10 mM CGA concentration after 30 minutes of incubation at 36° C. (FIG. 3C).

### Example 3: Characterisation of Recombinant IbGDSL Activity in P. pastoris Culture Supernatant

The bioconversion of CGA into 3,5-DCQ in vivo requires the establishment of a highly metabolically active P. pastoris culture. The yeasts are cultured in nutrient medium containing a 100 mM potassium phosphate buffer with a pH adjusted to pH 6.0. This allows the medium in which the enzyme obtained from the microbial cells and the substrate will be in contact, to be maintained at pH 6. After establishing an inoculum of 25 mL of yeast culture overnight at 30° C. under agitation of 250-300 rpm, this is used to inoculate a volume of 100-200 mL of nutrient medium in such a way as to obtain an OD₆₀₀ₙₘ around 1. To this culture 0.5% methanol is added in order to initiate the production of IbGDSL. The addition of methanol is repeated every 24 hours to maintain the level of induction of protein production. Three days after the establishment of this culture, CGA is added directly to the medium to establish a final concentration of 10 mM.

The first step of this experiment is to show whether the IbGDSL produced by P. pastoris is capable of converting CGA into 3,5-DCQ knowing that the original enzyme could potentially be glycosylated three-fold and that the glycan trees generated by P. pastoris are not comprised and organised in the same way as the plant glycan trees. These differences could directly influence the stability and prevent the proper activity of the enzyme. To do so, supernatants from two P. pastoris cultures, one transformed with the empty vector (negative control) and the other with the vector carrying the gene encoding IbGDSL were recovered after 3 days of methanol induction. These supernatants were incubated at pH 6.5 with 10 mM chlorogenic acid for 30 minutes at 36° C. The enzymatic reaction was then stopped with the addition of ethanol and analysed by UPLC-MS.

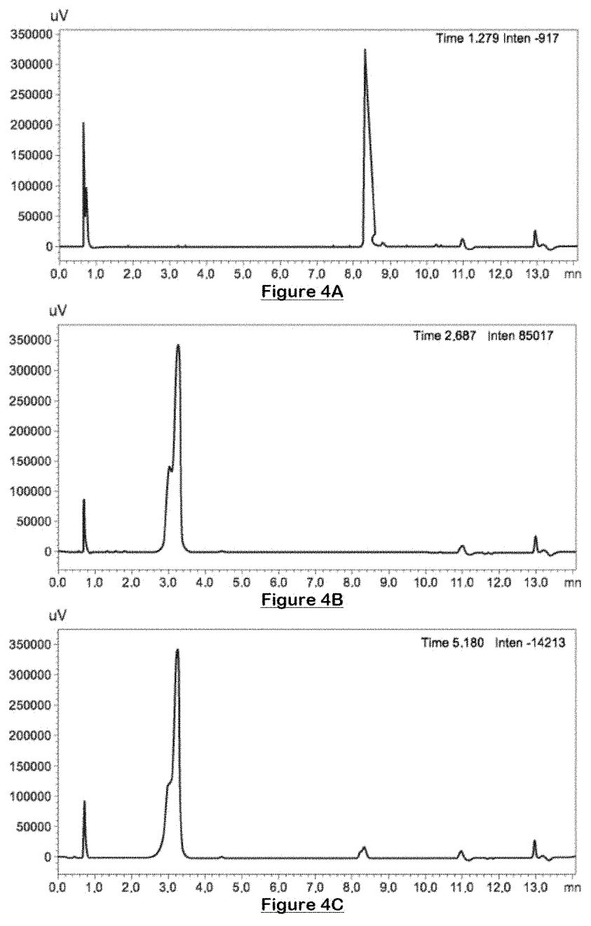

The results obtained show that unlike the negative control (FIG. 4B), the supernatant in which IbGDSL was secreted shows a peak with the same retention time (8.3 min) and mass (m/z neg 515) (FIG. 4C) as the 3,5-DCQ standard (FIG. 4A). The enzyme synthesised by this microbial heterologous expression system is therefore functional despite some differences at the post-translational level. It should be taken into account that the incubation lasted only 30 minutes which could explain the low intensity of the peak.

The second step of this experiment is to show whether the CGA directly added to the P. pastoris culture medium expressing the enzyme could be directly converted into 3,5-DCQ. The objective is to avoid the purification step of the enzyme if necessary.

After inducing protein expression with methanol for 3 days, the CGA was added directly to the culture buffered at pH 6 at a final concentration of 10 mM and left in contact with the microbial cells for 3 days. This experiment was conducted at 30° C., the ideal temperature for the culture and growth of the P. pastoris organism.

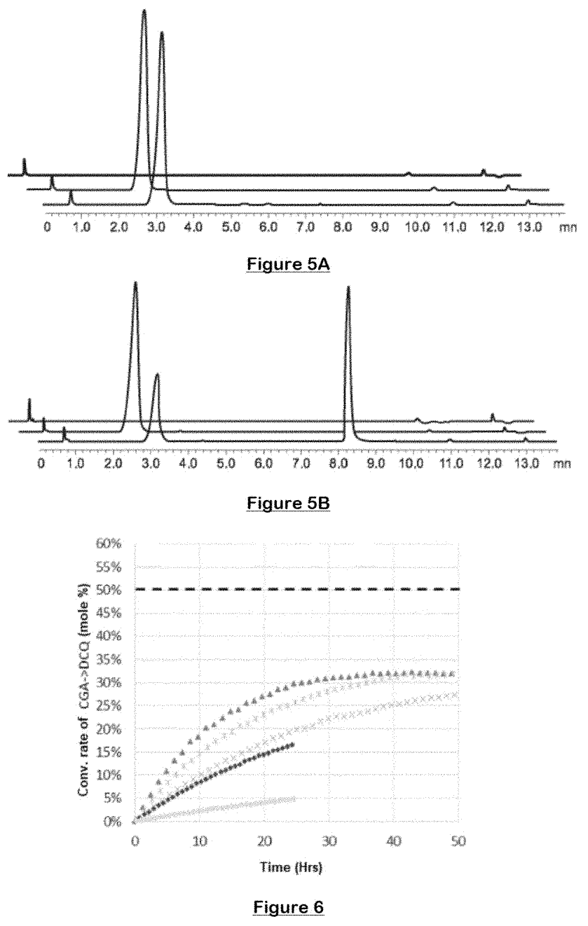

The supernatant was then analysed by UPLC-MS. The results obtained set forth in FIG. 5B show that the culture of P. pastoris expressing IbGDSL is able to convert CGA into 3,5-DCQ within 3 days with a very high efficiency. Indeed, a bioconversion yield of around 44% was estimated, the maximum stoichiometric limit being 50% since it takes two molecules of CGA to form one molecule of 3,5-DCQ.

It has been demonstrated that CGA can be added indifferently every day, or at the beginning of the induction phase, or at the end of the induction phase, without affecting the final conversion rate obtained.

The conversion kinetics of pure CGA into 3,5 CDQ were established (FIG. 6A) as a function of the starting CGA concentrations. After 50 hours of bioconversion, a maximum yield of 32% was obtained for both 5- and 7.5-mM concentrations. The concentrations above 7.5 mM tested here degrade the final yields of 3,5-DCQ.

The 3,5-DCQ contents measured after 50 hours of bioconversion show maximum amounts in the order of 1.2 g/L for starting pure CGA concentrations of 7.5 and 9 mM (FIG. 6B).

### Example 4: Bioconversion of Chlorogenic Acid from Green Coffee Extract to 3,5-DCQ by the Culture Supernatant of P. pastoris Cells Expressing IbGDSL, Secreted into Said Culture Medium

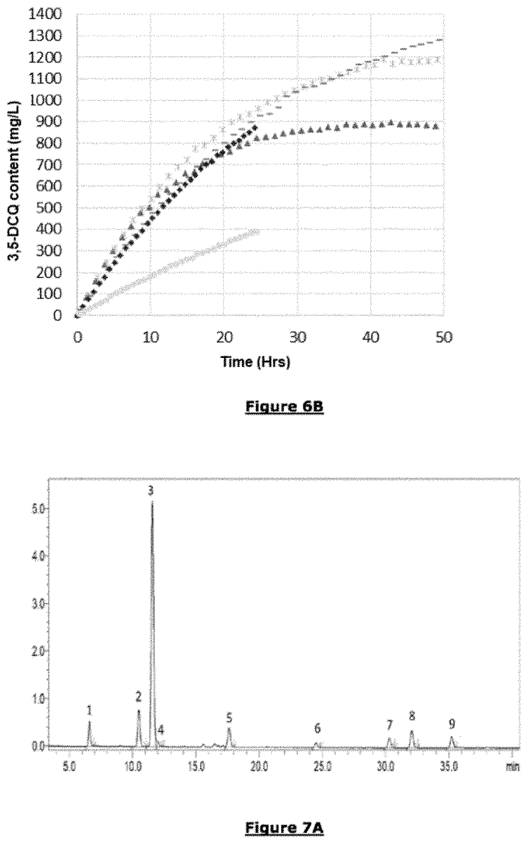

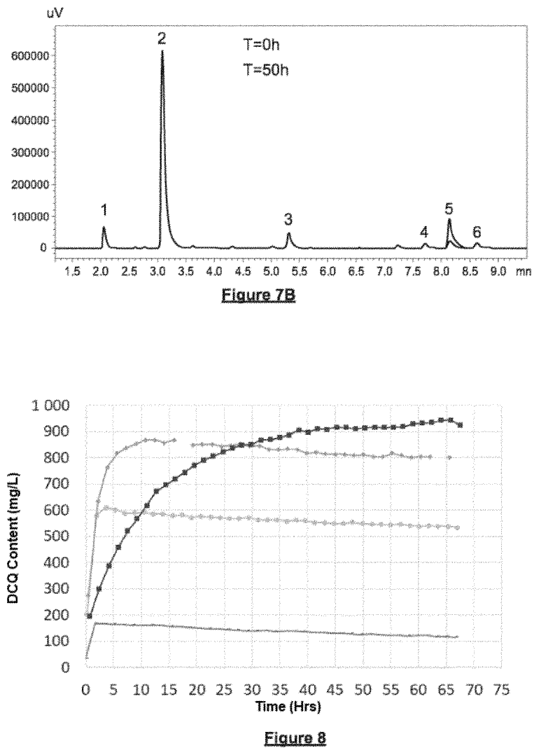

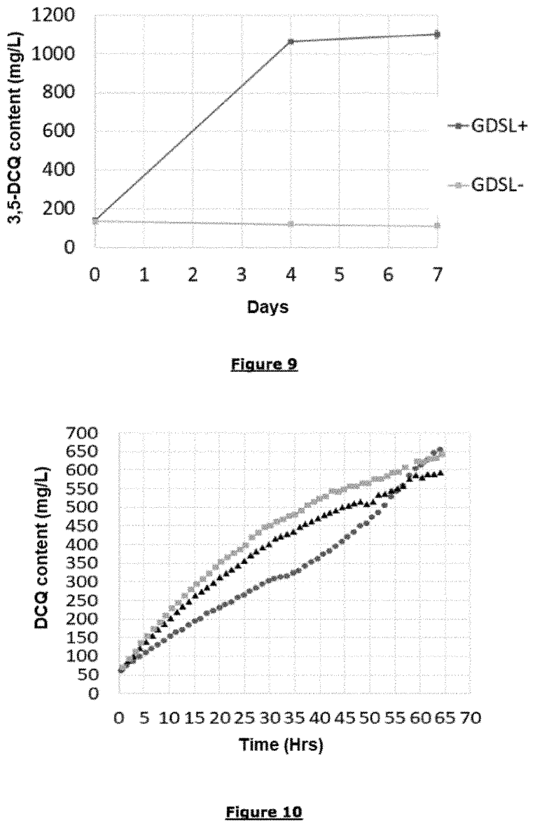

The bioconversion reaction of CGA into 3,5-DCQ was also carried out from a green coffee (Coffea canephora) extract whose composition is indicated in FIG. 7A. This green coffee extract, comprising a CGA concentration equivalent to 10 mM and bioconverted over a period of 50 hours, at 30° C. and pH 6, led to a new extract whose composition is shown in FIG. 7B. It is noticed that IbGDSL exclusively catalyses the formation of 3,5-DCQ, to the exclusion of any other isomer. Furthermore, no other caffeic acid-containing substrate than chlorogenic acid is biotransformed as no decrease in peaks is observed except for that corresponding to CGA. Bioconversion of green coffee extract with IbGDSL increases the 3,5-DCQ content initially present in the extract 4.5-fold (200 mg/L at T₀ and 900 mg at T₅₀ₕ) for a starting CGA concentration equivalent to 10 mM. Bioconversion of CGA into 3,5-DCQ from the green coffee extract was performed after 4 and 7 days, in order to analyse the effect of a reaction with GDSL taking place over a long period of time (FIG. 9). It is noticed that the 3,5-DCQ content measured does not increase between day 4 and day 7 and therefore there is no particular interest in carrying out long fermentations. The concentration of the IbGDSL enzyme obtained by fermentation of P. pastoris accelerates the reaction speed of the conversion of CGA into 3,5-DCQ (FIG. 10). It is noticed that the same level of 3,5-DCQ concentration can be obtained for enzyme concentration factors of 10-, 20- and 37-fold after 60 h of bioconversion. In conclusion, the recombinant IbGDSL enzyme obtained from P. pastoris cultures is an efficient catalyst to obtain the conversion of chlorogenic acid into 3,5-DCQ in large amounts, either by converting pure chlorogenic acid or by transforming a plant extract naturally containing chlorogenic acid such as a green coffee extract.

1-14. (canceled)

15. A protein comprising, or consisting of, an amino acid sequence selected from:
SEQ ID NO: 1,
a sequence having at least 80% identity with SEQ ID No. 1,
a fragment including at least 50 amino acids of said SEQ ID No. 1 and
a fragment including at least 50 amino acids of said sequence having at least 80% identity with SEQ ID No. 1,
said protein being capable of converting chlorogenic acid into isochlorogenic acid.

16. The protein according to claim 15, wherein said protein is selected from GDSL esterase/lipase enzymes, in particular a GDSL esterase/lipase enzyme of Ipomoea.

17. The protein according to claim 16, wherein said protein is selected from the GDSL esterase/lipase enzymes of Ipomoea batatas.

18. An isolated nucleic acid molecule encoding a protein according to claim 15.

19. The isolated nucleic acid molecule according to claim 18, said molecule comprising or consisting of a nucleic acid sequence selected from: SEQ ID No. 2 and a sequence having at least 80% identity with SEQ ID No. 2.

20. A recombinant vector comprising at least one nucleic acid molecule according to claim 18, each of said at least one molecule being placed under the control of means necessary for the expression of said protein in a host cell.

21. A host cell comprising:
at least one isolated nucleic acid molecule according to claim 18, or
at least one recombinant vector comprising said at least one isolated nucleic acid molecule, each of said at least one molecule being placed under the control of means necessary for the expression of said protein in a host cell.

22. The host cell according to claim 21, wherein said host cell is selected from yeast cells, in particular of the Pichia pastoris strain, or from plant cells, in particular Nicotiana benthamiana.

23. A method for producing isochlorogenic acid comprising contacting, under appropriate reaction conditions, chlorogenic acid and a protein comprising, or consisting of, an amino acid sequence selected from:
SEQ ID NO. 1,
a sequence having at least 80% identity with SEQ ID No. 1,
a fragment including at least 50 amino acids of said SEQ ID No. 1, and
a fragment including at least 50 amino acids of said sequence having at least 80% identity with SEQ ID No. 1,
said protein being capable of converting chlorogenic acid into isochlorogenic acid, to obtain an isochlorogenic acid-enriched composition.

24. The method according to claim 23, further comprising:
a prior step of culturing, in an appropriate culture medium and under appropriate conditions, a host cell capable of expressing a protein comprising, or consisting of, an amino acid sequence selected from: SEQ ID No. 1, a sequence having at least 80% identity with SEQ ID No. 1, a fragment including at least 50 amino acids of said SEQ ID No. 1 and a fragment including at least 50 amino acids of said sequence having at least 80% identity with SEQ ID No. 1, said protein produced being optionally purified, and/or said chlorogenic acid is present in isolated form or in a composition, said composition being especially a plant extract, and/or
a subsequent step of isolating the isochlorogenic acid produced during the reaction.

25. The method according to claim 23, wherein:
said protein comprising, or consisting of, an amino acid sequence selected from SEQ ID No. 1, a sequence having at least 80% identity with SEQ ID No. 1, a fragment including at least 50 amino acids of said SEQ ID No. 1 and a fragment including at least 50 amino acids of said sequence having at least 80% identity with SEQ ID No. 1, is produced and then exported in the culture supernatant by P. pastoris cells, and
said chlorogenic acid is present in a composition which is a green coffee extract.

26. A product likely to be obtained by a method according to claim 23, said method comprising contacting, under appropriate reaction conditions, a green coffee extract whose chlorogenic acid concentration is greater than or equal to 2 mM, and a protein capable of converting chlorogenic acid into isochlorogenic acid and comprising, or consisting of, an amino acid sequence selected from:
SEQ ID NO. 1,
a sequence having at least 80% identity with SEQ ID No. 1,
a fragment including at least 50 amino acids of said SEQ ID No. 1 and
a fragment including at least 50 amino acids of said sequence having at least 80% identity with SEQ ID No. 1.

27. The product according to claim 26, said method comprising the following steps:
a step of culturing P. pastoris transformed by an expression vector having integrated the gene encoding the IbGSDL protein of SEQ ID No. 1, in an appropriate nutrient medium and under appropriate conditions, said protein being produced and then exported in the culture supernatant,
optionally purifying the protein of SEQ ID No. 1, and
contacting, under appropriate reaction conditions, a green coffee extract whose chlorogenic acid concentration is greater than or equal to 2 mM, with said culture supernatant, or optionally with said purified protein,
optionally a subsequent step of isolating the isochlorogenic acid produced during the reaction.

28. A method of converting chlorogenic acid into isochlorogenic acid, comprising:
adding to an extract comprising chlorogenic acid at least one protein, said protein comprising, or consisting of an amino acid sequence selected from:
SEQ ID NO. 1,
a sequence having at least 80% identity with SEQ ID No. 1
a fragment including at least 50 amino acids of said SEQ ID No. 1 and
a fragment including at least 50 amino acids of said sequence having at least 80% identity with SEQ ID No. 1,
said protein being capable of converting chlorogenic acid into isochlorogenic acid, or a host cell expressing such a protein, to convert chlorogenic acid into isochlorogenic acid.
